(19) United States
(12) Patent Application Publication
(10) Pub. No.: US 2014/0147802 A1
(43) Pub. Date: May 29, 2014

(54) Device for Dentistry Treatments

(71) Applicant: Moreno Naldoni, Scandicci (IT)

(72) Inventor: Moreno Naldoni, Scandicci (IT)

(73) Assignee: GP INVESTIMENTI S.r.l., Montespertoli (IT)

(21) Appl. No.: 14/112,258

(22) PCT Filed: Apr. 20, 2012
(86) PCT No.: PCT/IB2012/052014

§ 371 Date: Dec. 26, 2013

(30) Foreign Application Priority Data
Apr. 21, 2011 (IT) BO2011A000216

(51) Int. Cl. A61B 5/00; A61M 13/00; A61B 5/01; A61C 1/08; A61B 18/20; A61N 5/06

(52) U.S. Cl. CPC A61B 5/0088; A61B 18/20; A61N 5/062; A61N 5/0603; A61B 5/4836; A61B 5/01; A61B 5/0071; A61C 1/088; A61M 13/003; A61B 5/742
USPC 433/27; 433/29

(57) ABSTRACT

Device for dentistry treatments having a photodiagnosis unit for performing photodiagnosis of a portion of gingival tissue, a temperature measurement unit for measuring the temperature of the portion of gingival tissue, a photoablation unit for performing photoablation of part of the portion of gingival tissue, a phototherapy unit for performing photodynamic and/or photoinductive therapy of the portion of gingival tissue, a control unit configured to acquire, by means of the photodiagnosis unit and the temperature measurement unit, information relative to the state of the portion of gingival tissue, and to control operation of the photoablation and phototherapy units according to the information acquired.

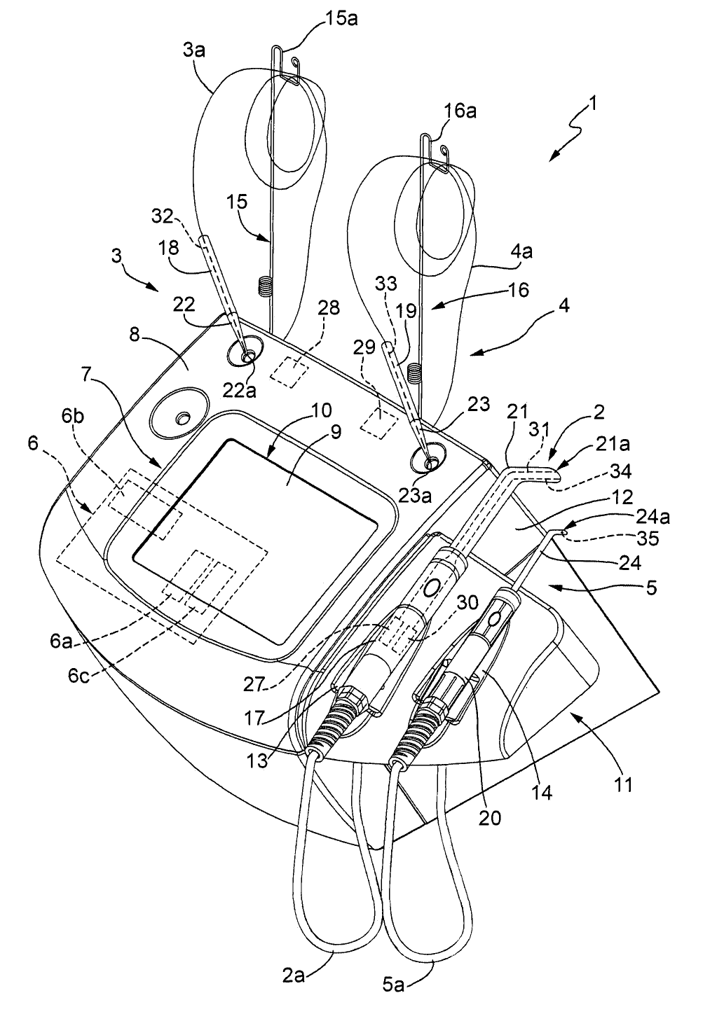

### TECHNICAL FIELD

The present invention concerns a device for dentistry treatments.

In particular, the present invention is advantageously but not exclusively applied in the treatment of periodontitis, and in particular for treatment of the inflammatory state of the periodontal soft tissues, such as gums and mucous membranes, to which the following description will explicitly refer without loss of generality.

### BACKGROUND ART

Periodontitis, also commonly known as pyorrhea, is an inflammatory pathology of the periodontal tissues which currently represents, in countries with a high standard of living, the main cause of tooth loss and disorders correlated with malocclusion. Periodontitis affects 50% of the adult population in the moderate form and between 5% and 15% in the severe form. The disease mechanism of periodontitis is multifactorial, but the determining factors include colonisation of the periodontal tissue by pathogenic germs including, first and foremost, Porfiromonas gingivalis and Actinobacyllus actinomycetemcomitans.

In recent years, a causal relation has been ascertained between chronic bacterial colonisation of the periodontium and the incidence of cardiovascular diseases, such as atherosclerosis, cardiac ischemia, ictus and peripheral obliterative arteriopathies. Chronic periodontitis constitutes a per se risk factor, added to those connected with lifestyle. Studies have demonstrated that patients with periodontitis have a significantly higher probability of contracting cardiovascular diseases, varying from 25% to 70%. The pathogenetic link consists in release into the circulatory system of bacteria or their toxic and pro-inflammatory products, such as lipopolysaccharides, prostanoids and cytokines, which induce a state of distress of the blood vessel tissues, a precursor to cardiovascular disease. The release into circulation of pro-inflammatory factors from the chronic periodontal centres of infection can occur intermittently in relation to various events, such as mastication, oral hygiene operations, dental surgery or the therapy normally used in the treatment of periodontitis. Lastly, it is interesting to note that, according to recent data of the Ministry of Health, cardiovascular diseases represent the main cause of 44% of all deaths currently registered in Italy. Looking ahead, in 10 years it is calculated that there will be over 240,000 cardiovascular pathology cases per year unless the relative risk factors are successfully reduced.

The traditional treatments for periodontitis are based on a combination of mechanical, antiseptic and antibiotic treatments.

The mechanical treatments consist fundamentally in cleaning of the exposed surface of the tooth roots. These treatments are, however, in themselves inadequate for complete removal of the bacteria from the infected tissues, which are partly anatomically inaccessible to the devices used, and can cause bacteremic infections and also lesions of the cementum covering the root, exposing the dentinal tubules to bacterial colonisation. Furthermore, the parodontopathogenic bacteria have developed the strategy of invading the sulcular and coronal margins of the junctional epithelium to evade the defences of the host immune system and resist the traditional pharmacological therapies.

The antiseptic or antibiotic treatments, which are grouped into topical, for example chlorhexidine, or systemic, for example metronidazole, are designed to eliminate the bacterial load, but are not without problems as they can induce bacterial resistances and alterations of the normal buccal and gastrointestinal bacterial flora and can damage the periodontal tissue cells. By way of example, chlorohexidine, which represents the commonest topical treatment for periodontal diseases due to its high bactericidal activity vis-à-vis oral germs, can produce, at the concentrations normally used in clinical practice, lesions of the oral tissues.

When periodontitis is not adequately and promptly treated, it can also lead to edentulism, i.e. partial or total tooth loss, for which implantology is currently an important therapy. Dental implants have made great progress in recent years in terms of surgical techniques and implant materials and offer increasing guarantees of biocompatibility and duration in the long term. Among the most common complications of implant therapy is colonisation of the implant with the germs of the oral bacterial flora, including the micro organisms involved in periodontitis. Even when an adequate antisepsis of the implant is obtained, it is known that the metallic surface of the latter is able to adsorb products of bacterial degradation, such as the lipopolysaccharide (LPS) of the Gram-wall, causing conditions of chronic inflammation which can prejudice osteogenesis and, therefore, compromise osseointegration and strength of the implant.

From the above description, it is evident that there is a great need for new therapeutic strategies for the treatment of periodontitis and/or post-implant complications which can prevent release into the circulation of germs and proinflammatory factors, so as to reduce the risk of developing cardiovascular diseases.

### DISCLOSURE OF INVENTION

The object of the present invention is to produce a device for dentistry treatments, which allows efficient treatment of periodontitis, is free from the drawbacks described above and, at the same time, is easy and inexpensive to produce.

In accordance with the present invention a device for dentistry treatments and a method for treatment of the inflammatory state of at least one portion of gingival tissue are provided, as defined in the attached claims.

### BRIEF DESCRIPTION OF THE DRAWING

For a better understanding of the present invention, a preferred embodiment is now described, purely by way of non-limiting example and with reference to the single FIGURE attached, which illustrates the device for dentistry treatments produced according to the precepts of the invention.

### BEST MODE FOR CARRYING OUT THE INVENTION

In the FIGURE, 1 indicates, as a whole, the device for dentistry treatments of the present invention. The device 1 comprises a photodiagnosis unit 2 for performing a photodiagnosis of at least one portion of the gingival tissue of a patient (not illustrated), a photoablation unit 3 for performing a photoablation of at least part of said portion of the gingival tissue, a phototherapy unit 4 for performing a photodynamic therapy or a photoinductive therapy (photostimulation) of said portion of the gingival tissue and a temperature measurement unit 5 for measuring, without contact, the temperature of the portion of gingival tissue during the treatment.

The device 1 furthermore comprises an electronic control unit 6, which is configured to acquire data on the state of the portion of gingival tissue provided by the photodiagnosis unit and/or temperature data of the portion of gingival tissue provided by the temperature measurement unit 5, and to control operation of the photoablation unit 3 and the phototherapy unit 4 according to the data acquired, and a man-machine interface 7, which communicates with the control centre 6 and has the purpose of allowing an operator to give commands to the device 1 and display data and parameters relative to the dentistry treatment in progress. Each of the units 2, 3, 4 and 5 comprises a respective electrical and/or optical cable 2a, 3a, 4a and 5a connected to the control unit 6.

The device 1 comprises a main box-shaped structure 8 to contain and/or support the components of the device 1. In particular, the control unit 6 is housed inside the main box-shaped body 8 and the man-machine interface 7 comprises a touch-screen device 9, which is mounted at the level of a window 10 of the main box-shaped body 8, and other safety devices not illustrated such as key selectors and emergency switches. According to an alternative embodiment not illustrated, the man-machine interface 7 comprises, in place of the touch-screen device 9, a display unit mounted at the level of the window 10 and an alphanumerical keyboard.

The main box-shaped body 8 comprises a supporting appendix 11 protruding from a lateral wall 12 of the main box-shaped body 8 and having seats 13 and 14 in which it is possible to rest the photodiagnosis unit 2 and the temperature measurement unit 5 respectively. The device 1 comprises two elastic supports 15 and 16 consisting of respective rod irons with small diameter having respective first ends fixed to the main box-shaped body 8 and respective second ends 15a and 16a bent in a U shape and facing upwards, from which to hang, wound up, the cable 3a of the photoablation unit 3 and the cable 4a of the phototherapy unit 4 respectively. The main box-shaped body 8 can be installed on an appropriate trolley or on a dentist's unit, known per se and therefore not illustrated.

Each of the units 2, 3, 4 and 5 comprises a respective handpiece 17, 18, 19, 20 having a substantially cylindrical form which can be easily gripped by the operator and provided, at one first end, with a respective probe 21, 22, 23, 24. The cable 2a, 3a, 4a, 5a of each of the units 2, 3, 4 and 5 protrudes from the second end of the respective handpiece 17, 18, 19, 20. The seats 13 and 14 of the supporting appendix 11 are shaped to receive the body of the handpiece 17 of the photodiagnosis unit 2 and the body of the handpiece 20 of the temperature measurement unit 5 respectively. The main box-shaped body 8 has, in a portion near the fastening points of the elastic supports 15 and 16, two depressions 25 and 26 to receive the tip of the probe 22 and the tip of the probe 23 respectively when the respective handpieces 18 and 19 are the respective cables 3a e 4a hanging at the ends 15a and 16a of the elastic supports 15 and 16.

The photodiagnosis unit 2, the photoablation unit 3 and the phototherapy unit 4 comprise respective optical sources 27, 28 and 29 which are able to emit electromagnetic radiations generically in the optical spectrum, i.e. having wavelengths between part of the ultraviolet spectrum and part of the infrared spectrum, and are controlled by the control unit 6. The optical sources 28 and 29 of the photoablation unit 3 and the phototherapy unit 4 respectively are arranged inside the main box-shaped body 8. The optical source 27 of the photodiagnosis unit 2 is arranged inside the respective handpiece 17. Each unit 2, 3 and 4 comprises a respective optical conveying system to convey the radiations emitted by the respective optical source 27, 28, 29 outside the respective probe 21, 22, 23 at the level of a respective output hole 21a, 22a, 23a. In use, the probes 21, 22 and 23 are positioned, each during one or more dentistry treatment phases, with the respective output holes 21a, 22a, 23a near the portion of gingival tissue to be treated.

The photodiagnosis unit 2 furthermore comprises an optical receiver 30 able to detect optical electromagnetic radiations which, in use, are reflected or emitted by fluorescence by the portion of gingival tissue when it is struck by the radiation of the optical source 27. The optical receiver 30 is also housed inside the handpiece 17.

The optical source 27 of the photodiagnosis unit 2 emits electromagnetic radiations having a first wavelength L2 selected in the interval ranging from 350 to 450 nm. Advantageously, the wavelength L2 is equal to 400±10 nm, i.e. it falls within the interval ranging from 390 to 410 nm. Therefore, the optical source 27 is a visible violet light source. The optical source 27 consists, for example, of a wide spectrum lamp provided with a filter to obtain the wavelength L2, or a LED source or a laser source able to directly emit the wavelength L2. The optical receiver 30 of the photodiagnosis unit 2 consists, for example, of a CCD image sensor, or a photodiode array, or a small video camera, or a spectrometer device, or a spectrophotometer device.

The control unit 6 comprises electronic circuits 6a for processing, in analog form, the signal provided by the optical receiver 30 and a microcontroller 6b for acquiring and processing the signal processed by the electronic circuits 6a so as to obtain information on the inflammatory state and on the level of bacterial contamination of the portion of gingival tissue and on the actual part of tissue removed by the photoablation.

According to a further embodiment, the optical receiver 30 comprises several coupled detector devices, for example a video camera and a spectrometer. In this way, the microcontroller 6b is able to obtain further information, such as the type of gingival tissue being treated, for example whether it is an epithelial tissue or a connective tissue.

The optical source 28 of the photoablation unit 3 emits electromagnetic radiations having a second wavelength L3 selected in the interval ranging from 780 nm to 1200 nm, and preferably in the sub-interval ranging from 800 to 850 nm. Advantageously, the wavelength L3 is equal to 810±10 nm, i.e. it falls within the interval ranging from 800 to 820 nm. Therefore, the optical source 28 is an infrared radiation source.

The optical source 29 of the phototherapy unit 4 emits electromagnetic radiations having a third wavelength L4 selected in the interval ranging from 600 to 700 nm. Advantageously, the wavelength L4 is equal to 650±20 nm, i.e. it falls within the interval ranging from 630 to 670 nm. Therefore, also the optical source 29 is a source of visible red light.

Advantageously, the sources 27 and 28 are laser sources, and in particular consist of respective laser diodes. The optical source 29 consists of a LED or a laser diode. According to further embodiments, the source 27 consists of a high brilliance LED diode, or both the sources 27 and 28 consist of respective high brilliance LED diodes.

The optical conveying systems of the photoablation unit 3 and the phototherapy unit 4 each comprise a respective optical fibre 32, 33 which extends all along the respective handpiece 18, 19, from the output hole 22a, 23a of the respective probe 22 and 23. The handpieces 18 and 19 therefore consists of respective rigid fibre-holder casings. Each optical fibre 32, 33 has a diameter of between 200 and 600 μm and is made of silica/silica/polyimide. Each optical fibre 32, 33 extends, without interruption, as far as the respective optical source 28, 29, along and inside the respective cable 3a, 4a, which is optically shielded and is connected between the respective handpiece 18, 19 and the main box-shaped body 8.

According to a further embodiment not illustrated of the invention, each optical fibre 32, 33 is divided into two sections connected by means of an optical connector, a first section being arranged along the respective handpiece 18, 19, and a second section being arranged along the respective cable 3a, 4a.

According to a further embodiment not illustrated of the invention, the handpieces 18 and 19 comprise control buttons and signalling lights.

The optical conveying system of the photodiagnosis unit 2 comprises two optical guides 31 and 34 which extend along the probe 21, the first from the output hole 21a to the optical source 27 and the second from the output hole 21a to the optical receiver 30. The optical guide 31 is suitable for conveying to the outside of the probe 21 the optical radiation emitted by the optical source 27 and the other optical guide 34 is suitable for conveying towards the optical receiver 30 the optical radiation reflected, or emitted by fluorescence, by the portion of gingival tissue. The optical guide 31 has a diameter of between 8 and 12 mm to generate a light spot able to cover, in use, a sufficiently wide area of the portion of gingival tissue to be treated. The optical guides 31 and 34 consist, for example, of rigid optical guides made of silicate or borosilicate glass. According to a different embodiment, the optical guides 31 and 34 consist of two respective optical fibre bundles, the fibres of a first bundle being parallel to the fibres of the other bundle. The fibres of the optical guides 31 and 34 are made, for example, of silica/silica/polyimide, or of another plastic material. The photodiagnosis unit 2 comprises an electrical cable 2a to connect the optical source 27 and the optical receiver 30 to the control unit 6.

According to a further embodiment not illustrated of the invention, the device 1 comprises a fifth handpiece dedicated exclusively to detection of the optical radiations, i.e. the optical receiver 30 is housed inside said fifth handpiece.

According to a further embodiment not illustrated of the invention, the photodiagnosis unit 2 is similar to the units 3 and 4, i.e. it has the respective optical source 27 and the optical receiver 30 housed inside the main box-shaped body 8 and the optical conveying system of each of the units 2 and 4 and the optical guides 31 and 34 consist of two respective optical fibre bundles, which extend from the output hole 21a of the probe 21 to the respective source 27 and receiver 30, passing along the cable 2a, which therefore consists of a shielded optical cable.

As regards the temperature measurement unit 5, it comprises a temperature sensor 35 consisting, for example, of a thermoelectric sensor, or a photodiode, or a thermopile, or an infrared temperature sensor, or a thermocamera. The temperature sensor 35 is arranged at the level of the output hole 24a of the probe 24 of the temperature measurement unit 5. According to various embodiments, the temperature sensor 35 consists of a single sensitive element or an array of sensitive elements.

The control unit 6 comprises electronic circuits 6c for processing, in analog form, the signal provided by the temperature measurement unit 5. The microcontroller 6b is suitable for acquiring and processing the signal processed by the electronic circuits 6c so as to obtain temperature values of the portion of gingival tissue.

According to a further embodiment not illustrated of the invention, the photodiagnosis unit 2 and the temperature measurement unit 5 are at least partially integrated in one single handpiece provided with the two respective probes 21 and 24.

According to a further embodiment not illustrated of the invention, the device 1 comprises a skin cooling unit for blowing air onto the portion of gingival tissue so as to cool it during the treatment and/or reduce the pain caused by heating of the portion of gingival tissue. The skin cooling unit comprises an air delivery device, which comprises a handpiece provided with tubular probe to deliver air onto the portion of gingival tissue, and a valve system for connecting the probe to an air supply and distribution system outside the device 1. The valve system comprises a shut-off solenoid valve and a cock. Alternatively, if the external air supply and distribution system is not available, the delivery device comprises a small compressor or a blower housed inside the main box-shaped body 8 connected upstream of the valve system.

According to further embodiments not illustrated of the invention, the cooling unit probe is integrated in the handpiece 18 of the photoablation unit 3 or in the handpiece 20 of the temperature measurement unit 5.

The device 1 described above allows the performance of a particular method of treatment of the inflammatory state of at least one portion of gingival tissue, said treatment method constituting a further aspect of the present invention and comprising the stages described below.

The treatment method comprises an initial diagnosis stage to acquire data relative to the initial state of the portion of gingival tissue to be treated. In particular, the initial diagnosis stage comprises an initial photodiagnosis, which allows initial information to be acquired relative to the inflammatory state and the level of bacterial contamination of the portion of gingival tissue and the type of gingival tissue to be treated. The initial diagnosis stage furthermore comprises an initial temperature measurement, which allows acquisition of the initial temperature values of the portion of gingival tissue.

The photodiagnosis consists essentially in emitting, towards the portion of gingival tissue, optical radiations in the form of an optical radiation having wavelength L2, receiving the consequent radiation reflected or emitted by luminescence (fluorescence) by the portion of gingival tissue and determining the initial information mentioned above, relative to the initial state of the portion of gingival tissue, according to the radiation reflected or emitted by luminescence. Said information comprises data relative to the epithelial, connective and vascular architecture and the cellular component (for example polymorphonucleates, red globules, etc.) of the portion of gingival tissue. Furthermore, the part of the information relative to the inflammatory state allows evaluation of the cell vitality and analysis of the production of nitrogen oxide (NO), cytokines, prostaglandins and toxins released into the gingival tissue by the inflammatory process and by the bacteria.

In a subsequent photoablation phase, part of the portion of gingival tissue is removed according to the information acquired by the photodiagnosis, and in particular according to the information on the inflammatory state, on the level of bacterial contamination on the type of gingival tissue being treated, in order to remove all the inflamed tissue. The photoablation is performed by emitting, towards the portion of gingival tissue, optical radiations in the form of a laser radiation having the wavelength L3 to cause a selective ablation of intra- and extra-sulcular epithelial cells. If necessary, the photoablation stage comprises the application of an adjuvant substance, for example a scavenger substance with antioxidant and/or anti-inflammatory action on the gingival tissue before emitting the optical radiation with wavelength L3 which produces the ablation. The emission parameters of the optical radiation with wavelength L3, such as the continuous or pulsed emission mode and the emission power, are adjusted according to the information acquired by the initial photodiagnosis. A further positive effect of the photoablation is that it produces a coarctation of the blood vessels which open during the ablation.

At this point, a control or progress diagnosis phase is performed to acquire new data relative to the state of the gingival tissue after the photoablation in order to verify the effects of the ablation on the gingival tissue. The progress diagnosis procedure is analogous to that of the initial diagnosis. In particular, the progress diagnosis comprises a progress photodiagnosis and a progress temperature measurement. The new information acquired with the progress diagnosis comprises information analogous, in terms of type, to the information obtained from the initial diagnosis. Furthermore, the progress photodiagnosis allows the acquisition of information on the actual part of tissue removed by photoablation. For example, by using an orange optical filter, it is possible to see, from the violet light (wavelength L2) reflected by the portion of gingival tissue, the actual area of the portion of gingival tissue that has undergone the ablation. If the progress photodiagnosis reveals that not all the inflamed gingival tissue has been removed, then the photoablation is repeated a certain number of times until complete removal of the inflamed gingival tissue and remodelling of the gingival tissue. After each repetition of the photoablation the progress diagnosis is necessarily repeated. The progress temperature measurement, which is performed during or immediately after each single photoablation repetition, allows the temperature of the portion of gingival tissue to kept under control to avoid thermal damage to the gingival tissue. If necessary, if the temperature measured exceeds a certain temperature threshold, for example 60° C., the photoablation repetition is temporarily suspended.

If the progress diagnosis gives a positive result, i.e. it reveals that all the inflamed gingival tissue has been removed, then photodynamic therapy of the portion of gingival tissue is performed according to the last data acquired by the progress photodiagnosis to remove bacteria, toxic substances and inflaming substances from the portion of gingival tissue. In other words, the photodynamic therapy has an anti-bacterial, anti-inflammatory and anti-toxic action on the portion of gingival tissue being treated. The photodynamic therapy is performed by emitting, towards the portion of gingival tissue, optical radiations having the wavelength L4. The emission parameters of the optical radiation with wavelength L4, for example the continuous or pulsed emission mode and the emission power, are adjusted according to the last information acquired by the progress photodiagnosis.

In further detail, the photodynamic therapy consists essentially in applying, on the portion of gingival tissue to be treated, a bactericide substance photoactivatable by optical radiations having wavelength L4 and, subsequently, emitting the optical radiations with wavelength L4 onto the portion of gingival tissue to which the photoactivatable bactericide substance has been applied. The photoactivatable bactericide substance consists, for example, of methylene blue. Advantageously, the photodynamic therapy is performed with an optical emission power at least equal to 70 mW.

In place of or in addition to the photodynamic therapy, a photoinductive therapy (photostimulation) can be performed to induce a regeneration of the gingival, epithelial and connective tissue, after the photoablation. With the photoinductive therapy, the growth of the bone tissue can also be stimulated, to allow quicker healing in the case of implants and/or peri-implantitis. The photoinductive therapy is also performed by emitting optical radiations with wavelength L4. However, the photostimulating effect of the photoinductive therapy is obtained by emitting optical radiations with an intensity lower than the one necessary for the photodynamic action. Advantageously, the photoinductive therapy is performed with an optical emission power at least equal to 10 mW.

Lastly, a final diagnosis of the portion of gingival tissue is performed to acquire further data relative to the state of the gingival tissue at the end of the treatment, in order to verify the effects of the phototherapy on the gingival tissue. The procedure of the final diagnosis is substantially identical to that of the progress diagnosis. In particular, the final diagnosis comprises a final photodiagnosis and a final temperature measurement.

The device 1 allows performance of the treatment method of the gingival tissue described above. In fact, the various photodiagnosis phases are performed by means of the photodiagnosis unit 2 and the temperature measurement unit 5, the photoablation is performed by means of the photoablation unit 3 and the photodynamic therapy and/or the photoinductive therapy are performed by means of the phototherapy unit 4. More specifically, the microprocessor 6b of the control unit 6 is configured to implement a method for control of operation of the device 1, said control method being provided with the present invention and described below.

The microcontroller 6b is configured to obtain, on the basis of the signal provided by the photodiagnosis unit 2, information relative to the inflammatory state, the level of bacterial contamination and the type of tissue of the portion of gingival tissue, information on the actual part of tissue removed by the photoablation and, on the basis of the signal provided by the temperature measurement unit 5, temperature values of the portion of gingival tissue. The microcontroller 6b is programmed to process the information obtained relative to the portion of gingival tissue so as to determine the treatment to be performed. The treatment determined is proposed to the operator, displaying it on the touch-screen device 9.

Each treatment is defined by indications on what unit to enable, the photoablation unit 3 or the phototherapy unit 4, and by operating parameters of the unit 3, 4 to be enabled. The operating parameters comprise emission parameters of the optical radiations, such as the optical radiation emission mode, which can be selected from continuous or pulsed mode, the optical radiation emission power, the optical radiation wavelength value and, in the case of pulsed mode, the duty-cycle of the optical radiation impulses.

In particular, as regards the photoablation unit 3, in pulsed mode the duration of the impulse can be selected in an interval ranging from 0.02 to 2 ms and the impulse repetition frequency can be selected in an interval ranging from 200 to 5000 Hz. The emission power P3 is adjusted also according to the emission mode. In continuous mode, the emission power P3 can be selected in an interval ranging from 0.5 to 2.5 W. In pulsed mode, the emission power P3 can be adjusted so that the emission energy of each impulse can be selected in an interval ranging from 0.1 to 100 mJ. Advantageously, the duration of the impulse is between 40 and 60 μs, the impulse repetition frequency is between 4000 and 5000 Hz and the emission energy is between 0.2 and 0.4 mJ. The P3 emission power intervals given above avoid, in use, carbonisation of the optical fibre 32 and, in combination with the dimensions and material of the latter, allow effective ablation of the gingival tissue without causing thermal damage to the same.

As regards the phototherapy unit 4, the emission power P4 can be selected in an interval ranging from 5 to 200 mW. In the case of photodynamic therapy, the emission power P4 can be selected in an interval ranging from 100 to 200 mW. In the case of photoinductive therapy, the emission power P4 can be selected in an interval ranging from 5 to 50 mW.

The control unit 6 acquires, via the touch-screen device 9, operator confirmation of the treatment proposed, enables the unit 3, 4 as scheduled by the treatment proposed and sets the operating parameters of the treatment proposed on the unit 3, 4 enabled. At this point the operator can activate the unit 3, 4 enabled and perform the treatment.

The microcontroller 6b is configured to inhibit activation of the unit 3, 4 currently enabled if the temperature value measured exceeds a pre-set temperature threshold, for example 60° C. In this way, when heating of the portion of gingival tissue being treated becomes excessive, the treatment in progress is suspended.

According to a further embodiment of the invention, the microcontroller 6b is configured to vary, if the temperature value measured exceeds the pre-set temperature threshold, one or more operating parameters of the unit 3, 4 currently enabled which has an effect on the energy transmitted to the portion of gingival tissue by the optical radiations emitted by the respective optical source 28, 29, so as to reduce the energy transmitted. Said parameters comprise, for example, the emission power P3, P4 of the optical radiations and/or the duty-cycle value and/or the value of the wavelengths L3 and L4. For example, the emission power P3, P4 could be reduced.

During performance of the treatment, the operator can interact with the device 1 via the man-machine interface 7 to analyse the data acquired with the photodiagnosis unit 2 and the temperature measurement unit 5, to carry out any manual adjustments of the operating parameters of the photoablation unit 3 and the phototherapy unit 4, for example also intervening manually on selection of the emission mode and on adjustment of the emission power, and to activate the photoablation unit 3 and phototherapy unit 4 so as to correctly perform the treatment.

The main advantage of the device 1 described above is to allow the performance of new treatments of the inflammatory state of the gingival tissue in semi-automatic mode to efficiently treat periodontitis and adequately treat post-implant complications following the application of a dental implant. In particular, the device 1 allows treatment of the inflammatory state of the gingival tissue described above; said treatment considerably inhibits the entry of bacteria, toxins and pro-inflammatory chemokines into the circulatory system and therefore considerably reduces the risk of developing cardiovascular diseases.

The device 1 described above can, therefore, be advantageously used for treatment of the inflammatory state of the soft tissues, such as gums and mucous membranes, for the treatment of peri-implantitis, since it is possible to perform a bactericide action (photodynamic therapy) and an action that stimulates re-growth of the epithelial and connective tissue and the bone (photostimulation), and as an instrument to assist in implantology, both for pre-implant disinfection treatment and/or sterilisation of the implant surfaces, and as a post-operative treatment to promote healing and reduce complications.

1. A device for dentistry treatments, comprising at least one photodiagnosis unit (2) for performing a photodiagnosis of at least one portion of gum tissue, at least one temperature measurement unit (5) for measuring the temperature of said portion of gum tissue, at least one photoablation unit (3) for performing a photoablation of at least part of said portion of the gum tissue, at least one phototherapy unit (4) for performing a photodynamic therapy and/or a photoinductive therapy of said portion of the gum tissue, processing and control means (6) configured to acquire, by means of said photodiagnosis unit (2) and said temperature measurement unit (5), information concerning the state of the portion of gum tissue and to control the operation of the photoablation and phototherapy units (3, 4) as a function of said information.

2. A device according to claim 1, wherein said photodiagnosis unit (2) comprises a first optical source (27) for emitting optical radiations having a first wave length (L2) selected within the interval ranging from 350 to 450 nm and optical receiver means (30) for detecting optical radiations which, in use, are reflected, or emitted by fluorescence, by said portion of gum tissue, when the latter is hit by the optical radiation emitted by said first optical source (27).

3. A device according to claim 1, wherein said photoablation unit (3) comprises a second optical source (28) for emitting optical radiations having a second wave length (L3) selected within the interval ranging from 780 to 1200 nm.

4. A device according to claim 3, wherein said photoablation unit (3) is suited to emit optical radiations in continuous or pulsed mode; in case of continuous mode, said second optical source (28) being suited to emit optical radiations with a first emission power (P3) selectable within an interval ranging from 0.4 to 2.5 W; in case of pulsed mode, said second optical source (28) being suited to emit optical radiations with an emission energy selectable within an interval ranging from 0.1 to 100 mJ.

5. A device according to claim 1, wherein said phototherapy unit (4) comprises a third optical source (29) for emitting optical radiations having a third wave length (L4) selected within the interval ranging from 600 to 700 nm.

6. A device according to claim 5, wherein said third optical source (29) is suited to emit optical radiations with a second mission power (P4) selectable within an interval ranging from 5 to 200 mW.

7. A device according to claim 2, wherein each one of said optical sources (27-29) consists of a respective laser diode.

8. A device according to claim 2, wherein each one of said first and second optical sources (27, 28) consists of a respective laser diode and said third optical source (29) consists of a LED.

9. A device according to claim 2, wherein each one of said second and third optical sources (28, 29) consists of a respective laser diode and said first optical source (27) consists of a LED.

10. A device according to claim 1, and comprising air skin cooling means for blowing air onto said portion of gum tissue, so as to cool it down during said photoablation and/or during said photodynamic therapy and/or during said photoinductive therapy.

11. A device according to claim 1, wherein said processing and control means (6) comprise processing means (6b), which are configured to obtain, based on the signal provided by said photodiagnosis unit (2), information concerning the inflammatory state and the bacterial contamination level of said portion of gum tissue and to enable one between the photoablation unit (3) and the phototherapy unit (4) and to set, as a function of said information, operating parameters of the unit (3, 4) enabled.

12. A device according to claim 1, wherein said processing and control means (6) comprise processing means (6b), which are configured to obtain, based on the signal provided by said temperature measurement unit (5), at least one temperature value of said portion of gum tissue and to inhibit the actuation of said photoablation unit (3) and/or of said phototherapy unit (4) in case said temperature value is higher than a predetermined temperature threshold.

13. A device according to claim 1, wherein said photoablation and phototherapy units (3, 4) comprise respective optical sources (28, 29) for emitting optical radiations; said processing and control means (6) comprising processing means (6b), which are configured to obtain, based on the signal provided by said temperature measurement unit (5), at least one temperature value of said portion of gum tissue and, in case said temperature value is higher than a predetermined temperature threshold, to vary at least one operating parameter of said photoablation unit (3) and/or of said phototherapy unit (4), so as to reduce the energy transmitted to said gum portion by the optical radiations emitted by the respective optical source (28, 29).

14. A treatment method of the inflammatory state of at least one portion of gum tissue, comprising:
performing an initial photodiagnosis of said portion of gum tissue, so as to acquire first items of information concerning the inflammatory state and the bacterial contamination level of said portion of gum tissue;
performing a photoablation as a function of said first items of information acquired, so as to remove the inflamed part of tissue of said portion of gum tissue;
performing a progress photodiagnosis of said portion of gum tissue after said photoablation, so as to acquire second items of information concerning the inflammatory state and the bacterial contamination level of said portion of gum tissue, as well as the actual part of tissue removed by means of said photoablation, in order to verify the effects of the photoablation on the gum tissue;
in case said progress photodiagnosis has a positive result, performing a phototherapy on said portion of gum tissue, so as to remove bacteria, toxic substances and inflammatory substances from the portion of gum tissue; and
performing a final photodiagnosis of said portion of gum tissue after said phototherapy, so as to acquire third items of information concerning the inflammatory state and the bacterial contamination level of said portion of gum tissue, in order to verify the effects of the phototherapy on the gum tissue.

15. A method according to claim 14, wherein each one of said initial photodiagnosis, progress photodiagnosis and final photodiagnosis comprises:
emitting, towards said portion of gum tissue, optical radiations having a first wave length (L2) selected within the interval ranging from 350 to 450 nm; and
receiving the consequent radiation, reflected or emitted by luminescence by said portion of gum tissue, so as to determine said items of information concerning the inflammatory state and the bacterial contamination level of said portion of gum tissue.

16. A method according to claim 14, wherein said photoablation step comprises:
emitting, towards said portion of gum tissue, optical radiations having a second wave length (L3) selected within the interval ranging from 780 to 1200 nm and first emission parameters adjusted as a function of said first items of information.

17. A method according to claim 14, wherein said phototherapy step comprises:
applying, on said portion of gum tissue, a photoactivatable bactericide substance which is activatable by means of optical radiations having a third wave length (L4) selected within the interval ranging from 600 to 700 nm;
emitting, towards the portion of gum tissue in which the photoactivatable bactericide substance has been applied, optical radiations having said third wave length (L4) and second emission parameters adjusted as a function of said second items of information.

18. A method according to claim 14, and comprising:
in case said control photodiagnosis has a negative result, repeating said photoablation;
immediately after the execution of each photoablation, acquiring temperature values of said portion of gum tissue; and
in case said temperature values are higher that a predetermined temperature threshold, suspending the repetition of said photoablation.
